(19) United States
(12) Patent Application Publication
(10) Pub. No.: US 2010/0305686 A1
(43) Pub. Date: Dec. 2, 2010

(54) LOW-PROFILE MODULAR ABDOMINAL AORTIC ANEURYSM GRAFT

(76) Inventors: Andrew H. Cragg, Edina, MN (US); Rodolfo C. Quijano, Laguna Hills, CA (US); Hosheng Tu, Newport Beach, CA (US); Stephen Sosnowski, Vista, CA (US); Robert J. Socci, JR., San Juan Capistrano, CA (US); George Wallace, Coto de Caza, CA (US)

Correspondence Address: Aaron J. Poledna;PERKINS COIE LLP, P.O. Box 1247, Seattle, WA, 98111-1247, US

(21) Appl. No.: 12/628,131

(22) Filed: Nov. 30, 2009

Related U.S. Application Data
(63) Continuation-in-part of application No. 12/466,044, filed on May 14, 2009.
(60) Provisional application No. 61/053,378, filed on May 15, 2008.

(51) Int. Cl. A61F 2/82

(52) U.S. Cl. 623/1.35

(57) ABSTRACT

Systems methods and devices address and ameliorate intralumenal aneurysms by excluding the same through endograft by pass techniques. Percutaneuous emplacement, use of improved aortic-stent assemblies and shotgun neck framing facilitates placement of modular graft sections, for example, to treat abdominal aortic aneurysms.

### CROSS-REFERENCE TO RELATED APPLICATIONS

This application is a continuation-in-part of U.S. patent application Ser. No. 12/466,044 filed May 14, 2009 which application claims the Paris Convention Priority benefit of U.S. Provisional 61/053,378 filed May 15, 2008.

### BACKGROUND OF THE INVENTIONS

1. Field of the Invention

The present inventions relate generally to modular bilumenal endograft systems for the treatment of abdominal aortic aneurysms. Specifically, the present invention includes systems for endovascular repair with percutaneously emplaced grafts disposed at optimized orientations in a primary stent assembly, or aortic cuff having a shotgun neck framework, inter alia.

2. Description of the Related Art

The aorta delivers blood and oxygen to all arterial branches of the body, and as such is the largest artery of the human body. The normal diameter of the thoracic aorta is in the order of about 3 cm at the tubular ascending portion, 2.5 cm at the descending thoracic aorta and 2 cm in the infrarenal abdominal aorta. The aortic dimensions vary relative to body surface area, age and gender with males typically having larger aortic dimensions than females.

An enlargement of the aorta beyond its normal diameter is termed an aneurysm and is generally a result of deterioration and weakness of the arterial wall. In the United States approximately 15,000 individuals a year die as a result of aneurysm rupture. If the aneurysm is diagnosed prior to rupture it can be repaired

The gold standard for aneurysm repair has long been surgical repair. This typically involves cutting open the dilated portion of the aorta and inserting a synthetic (Dacron or Gore-tex) tube. Once the tube is sewn into the proximal and distal portions of the aorta, the aneurysmal sac is wrapped around the artificial tube and sutured closed. Although effective surgical repair usually involves a 7-10 day post surgical hospital stay and several months of recovery.

In recent years, the endolumenal treatment of abdominal aortic aneurysms has emerged as a minimally invasive alternative to open surgery repair. In endovascular surgery, a synthetic graft (stent-graft consisting of a polyester or Teflon® tube inside a metal frame) is packaged within a catheter and the device is inserted, via a surgical cutdown, into the bloodstream through an artery in the leg. The catheter is guided to the desired location by the surgeon via X-ray visualization. Once in place, the graft is released from the catheter and expanded within the aneurysm sac. The stent-graft reinforces the weakened section of the aorta to prevent rupture of the aneurysm. The metal frame expands like a spring and holds the graft tightly against the wall of the aorta, cutting off the blood supply to the aneurysm. The blood now flows through the stent-graft and isolates the aneurysm. Endolumenal aneurysm treatment is generally more benign, resulting in a 1-2 day hospital stay and 1-2 week recovery.

During the past decade, numerous medical device companies have introduced endografts for the treatment of abdominal aortic aneurysms to the market. These include devices by Medtronic®, Gore®, Cook®, Endologix®, Cordis® and others. These devices are fabricated from surgical grade materials which are inherently thick and rigid by nature. Although clinically effective, the bulky construct of these devices require they be delivered through catheters 20 Fr or larger in diameter and require a surgical cutdown on the artery to be introduced. Although the cut-down approach significantly reduces patient recovery time and the acute complications that often accompany open surgical intervention, the ultimate goal and the market trend is to reduce the endograft and delivery system profile to enable the endograft to be delivered percutaneously thus eliminating the need for the cut-down procedure.

### SUMMARY OF THE INVENTION

Briefly stated, systems methods and devices address and ameliorate intralumenal aneurysms by excluding the same through endograft by-pass techniques. Percutaneuous emplacement, use of improved aortic-stent assemblies and shotgun neck framing facilitates placement of modular graft sections, for example, to treat abdominal aortic aneurysms.

According to embodiments there are disclosed bifurcated endografts for aneurysm treatment which comprise, in combination, systems that can be percutaneously delivered through a 12 Fr or smaller vascular introducer, further comprising at least an endograft element disposed within a primary stent assembly, or aortic cuff, which is tubular. Those skilled in the art readily understand the interchangeability of “primary stent assembly” with both aortic and tubular cuff and/or trunk, as coextensively defined throughout the instant specification.

According to embodiments, there is disclosed a modular two-piece abdominal aortic endograft system with “D-shaped” proximal ends and circular distal ends which can be axially aligned within the aorta; wherein each said modular piece is independently adjustable up and down relative to each other to accommodate the naturally anatomically variable orientation of the renal arteries.

According to embodiments, there is disclosed a method of constructing a modular quasi-customizable endovascular graft in situ, comprising in combination the steps of translumenally advancing a first deployment catheter to access the aorta deploying a tubular cuff within the aorta translumenally advancing a second deployment catheter to access the tubular cuff from the bottom deploying a first iliac graft within in the tubular cuff translumenally advancing a third deployment catheter to access the tubular cuff from the bottom and deploying a second iliac graft within the tubular cuff.

According to embodiments, there is disclosed a low-profile modular endograft system for comprising, in combination: a cuff and at least two endograft units, each endograft unit having a lumen, a proximal end and a distal end, wherein each endograft unit comprises a flexible tubular woven wire frame having a proximal end with a generally D-shaped cross-section configured to be secured at the cuff and a distal end having a generally circular cross section-configured to be placed and fixed in each of the iliac arteries, and a seal between each of said endograft units and said cuff

### BRIEF DESCRIPTION OF THE DRAWINGS

Additional objects and features of the present invention will become more apparent and the invention will be best understood from the following Detailed Description of the Invention, when read with reference to the accompanying drawings, wherein:

FIG. 1A shows a schematic depiction of aspects of the instant teachings, representing detailed structure of an exemplary D-graft, according to embodiments of the present invention;

FIG. 1B shows a partial cut-away view schematically depicting a pair of D-grafts with opposite charged magnets embedded in the facing surfaces of the two D-grafts, according to embodiments of the present invention;

FIG. 1C shows two grafts that are self-sealing even when placed asymmetrically, according to embodiments of the present invention;

FIG. 1D shows a pair of D-grafts with anchoring barbs, according to embodiments of the present invention;

FIG. 2A shows procedural steps for positioning a system for treating abdominal aortic aneurysms, according to embodiments of the present invention;

FIG. 2B shows procedural steps for positioning a system for treating abdominal aortic aneurysms, according to embodiments of the present invention;

FIG. 2C shows procedural steps for positioning a system for treating abdominal aortic aneurysms, according to embodiments of the present invention;

FIG. 3 is side elevational view of a primary stent assembly/aortic trunk component in accordance with the embodiments of present invention;

FIG. 3A is a cross sectional view taken along the line 3A-3A in FIG. 3, showing one configuration of a shotgun neck frame, according to the present invention, taken along the lines 3A-3A in FIG. 3;

FIG. 3B is a cross sectional view of an alternate configuration of a shotgun neck frame, according to the present invention, taken along the lines 3A-3A in FIG. 3;

FIG. 4 is an elevational perspective view of a first iliac segment in accordance with the embodiments of present invention;

FIG. 4A is a cross sectional view taken along the line 4A-4A in FIG. 4;

FIG. 4B is a cross sectional view taken along the line 4B-4B in FIG. 4;

FIG. 5 is a side elevational view of an assembled abdominal aortic aneurysm graft in accordance with the embodiments of present invention;

FIG. 5A is a cross sectional view taken along the line 5A-5A in FIG. 5;

FIG. 5B is a cross sectional view taken along the line 5C-5C in FIG. 5, according to embodiments;

FIG. 5C is a cross sectional view taken below the line 5C-5C in FIG. 5, according to embodiments;

FIG. 6 is a schematic representation of the vasculature in the vicinity of an abdominal aortic aneurysm, showing a deployment catheter positioned across the aneurysm via the ipsilateral iliac artery;

FIG. 7 is a schematic representation as in FIG. 6, showing a primary stent assembly/aortic trunk component partially deployed;

FIG. 8 is a schematic representation as in FIG. 7, showing a primary stent assembly/aortic trunk fully deployed;

FIG. 9 is a schematic representation as in FIG. 8, showing an ipsilateral iliac graft deployment catheter extending through the aortic trunk;

FIG. 10 is a schematic representation as in FIG. 9, showing an ipsilateral iliac graft fully deployed within the aortic trunk, and spanning the aneurysm;

FIG. 11 is a schematic view as in FIG. 10, showing a contralateral iliac deployment catheter in position through the aortic trunk;

FIG. 12 is a schematic illustration as in FIG. 11, showing an aortic trunk, and ipsilateral and contralateral iliac grafts fully deployed;

FIGS. 13A and 13B shows a detailed view of an embodiments of the present invention teachings, namely a primary stent assembly with shotgun neck framework.

### DETAILED DESCRIPTION

The present inventors have discovered that a device engineered for percutaneous placement having an introduction profile of at least about 12 Fr solves numerous problems in the art of endovascular grafting, particularly where bifurcated (split into at least two branches) and assembled modularly. Expressly incorporated herein by reference are U.S. Pat. and Publication Nos. 5,676,697; 6,383,193; 5,316,023; 5,078,726; 5,928,279; 5,897,587; 6,001,125; 6,004,348; 6,517,571; 6,786,920; 6,981,982; 6,808,533; 6,790,225; 2009/0182413; 2009/0173439; 2009/0036973; 2008/0208325; 2008/0114449; 2004/0162604; 2004/0054397.

The embodiments of the present invention described below relate particularly to a system for use in treating or repairing aneurysms. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below, as detailed herein and claimed as proprietary according to the instant teachings.

Systems for repairing abdominal and thoracic aortic aneurysms come in many forms. A typical system includes an anchoring and/or sealing component which is positioned in healthy tissue above the aneurysm and one or more grafts which are in fluid communication with the anchoring and/or sealing component. Essentially, the grafts are the components of the system that are utilized to establish a fluid flow path from one section of an artery to another section of the same or different artery, thereby bypassing the diseased portion of the artery. Essentially, the endovascular grafting system of the present invention comprises a number of components that make up a modular system. Although the overall scope of embodiments each comprises a number of components, the challenges associated with these types of systems include profile, flexibility and accessibility.

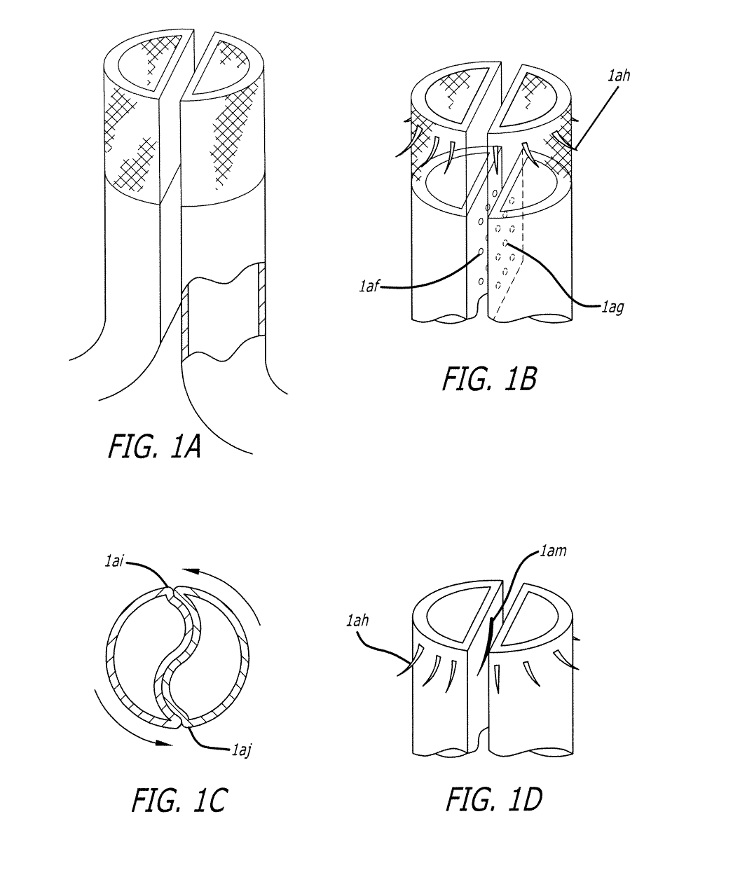

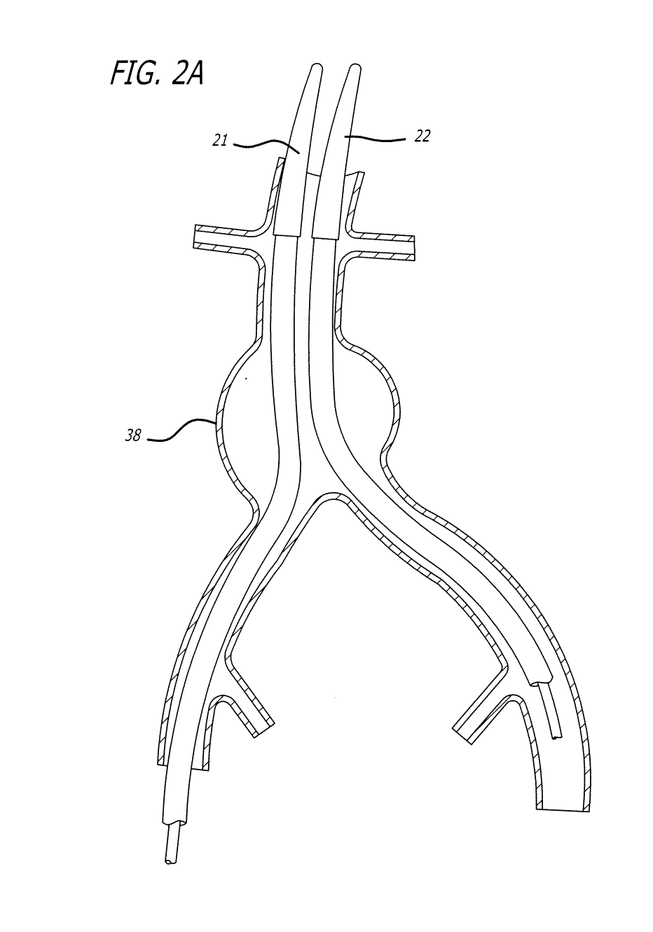

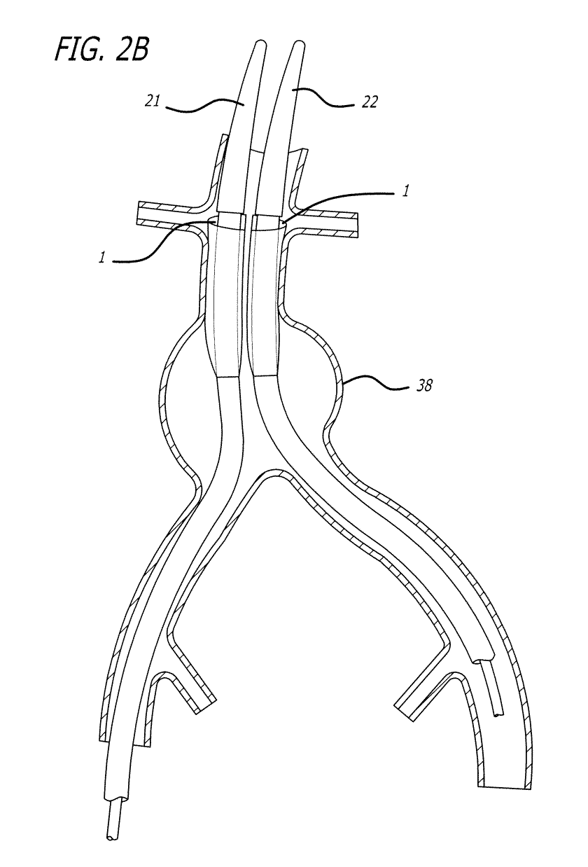

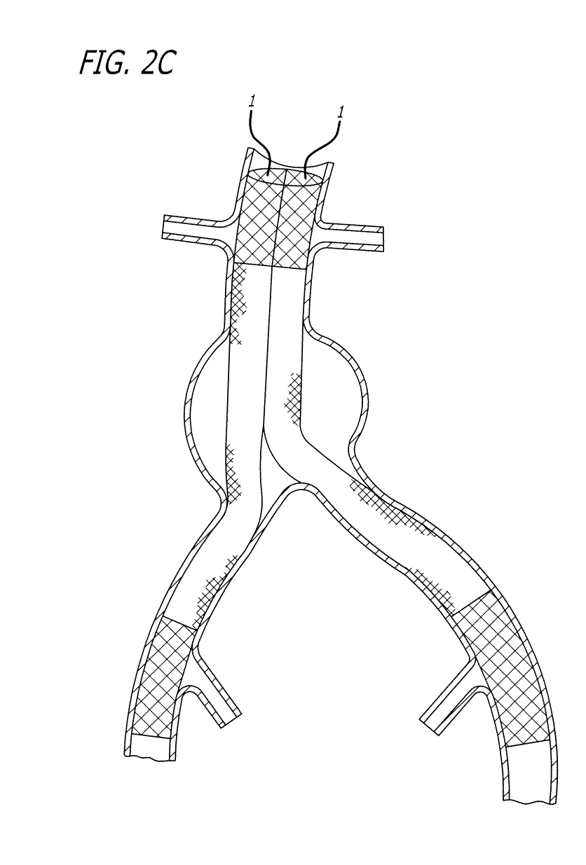

Referring now to FIGS. 1A-1D, various details of an exemplary D-shaped endograft are shown. Note also that FIGS. 2A-2C are demonstrative of proprietary delivery and construction systems for the present inventions. Those skilled in the art understand the schematic depictions represent teachings of the present invention for constructing modular grafts within an abdominal aortic aneurysm using deployment catheters 21, 22 to contact a pair of D-shaped grafts 1 (as shown the shown throughout); aortic aneurysm 38 is thus bridged creating a flow-path or lumen, which allows the aneurysm to shrink for want of blood flow.

According to the present invention, EVAR (endovascular aneurysm repair) of an abdominal aortic aneurysm with a stent graft includes features such as low introductory profiles, preferably 12 Fr or less, that expands up to 25 mm or more and can treat a short infrarenal neck, 15 mm long or less, which is constructed intralumenally from ultrathin graft materials attached to frames which provide structural support and enable the device to flex and conform to tortuous vessel anatomy.

According to embodiments, elements of a stent graft may comprise at least three layers, including a middle layer of a spiral wire or laser cut mesh of elastic or semi-rigid material (for example, metal, shape memory metal such as Nitinol®, plastic, shape memory plastic or other flexible expandable material), and an outer layer of ultrathin non-permeable expanded PTFE tape overwrap with a thickness of approximately 0.0005 inch. and a third inner layer of an ultrathin longitudinally stretchable expanded PTFE (polytetrafluoroethylene) tube of 0.004 inch or less, and/or dacron. The layers are thermally fused or bonded around the frame and serve as the building material for the stent graft composite. In embodiments, the expanded PTFE is impermeable to liquid or water. The inner PTFE layer and the outer PTFE layer serve to assure sufficient liquid-tightness of the composite constructing material to isolate the aneurysm from blood pressure. Alternatively, the graft material may also be an ultrathin tightly woven polyester fabric or like material 0.004 inch thick or less that is fastened to the frame with thread or glue at the proximal and distal ends and corrugated along the length to enable the graft to lengthen with the stent in the collapsed state and contract or shorten as the stent foreshortens during deployment.

According to embodiments, the mate-able pair of each D-graft set includes sides (as illustrated in FIG. 1A) which are manually maneuvered so they face each other. In one embodiment, at least a portion of the flat side of the grafts is embedded with rare-earth magnets with positive charge (1af) on one graft surface and negative charge (1ag) on the opposite graft surface to ensure control seal (for example, liquid-tight seal) and intimate contact of that portion when mating (FIG. 1B). In another embodiment, there is provided means for creating positive charged magnet at a first surface of the first graft and negative charged magnet at a second conformable surface of the second graft for intimate mating purposes. The conformable surface may be flat as in a D-graft. Those skilled understand the D-graft is meant to include any hemispheric shapes that would support the teachings of the present invention.

In another embodiment, barbs can be incorporated and spaced apart appropriately at about the proximal portion of the D-shaped graft so that the barbs (1ah) would be deployed radially outwardly to anchor the graft at the aorta in either the supra or infra renal positions or both (FIGS. 1B & 1D). In one embodiment, the barbs are generally sized and configured to allow the graft to move in an advancing direction with little resistance, whereas the barbs would engage into the aorta when the graft starts to move in a reversed direction. In another embodiment, the barbs are configured with a spring property so that the barbs extend outwardly (for example, spring-out) when the graft is deployed from the sheath. In still another embodiment, the barbs are made of shape memory material or temperature-sensitive material so that the barbs are activated at a threshold elevated temperature via hot saline or other electrical, chemical or biological means. In still another embodiment, the grafts are self-sealing or self-mating even when placed asymmetrically (FIG. 1C), wherein a portion of the contact surfaces mate against each other. The grafts as shown in FIG. 1C may comprise a pair of formed tube grafts or other radially expandable grafts that result in an intimate seal at the region between the two points (1ai and 1aj). The intimate seal region may be at about the proximal ends of the grafts or at proximity distal to the proximal ends. The grafts may be oversized so to intimately contact the arterial wall to seal the grafts and prevent blood leakage (endoleak).

According to embodiments the distal segment of each D-shaped portion 1 of the stent graft has a bare stent segment of approximately 25 mm length which is not covered by graft material FIG. 1A. This segment is placed across the renal arteries to enable supra renal fixation with barbs (1ah). The non-covered segment within the stent enable blood flow into the renal arteries FIG. 2C.

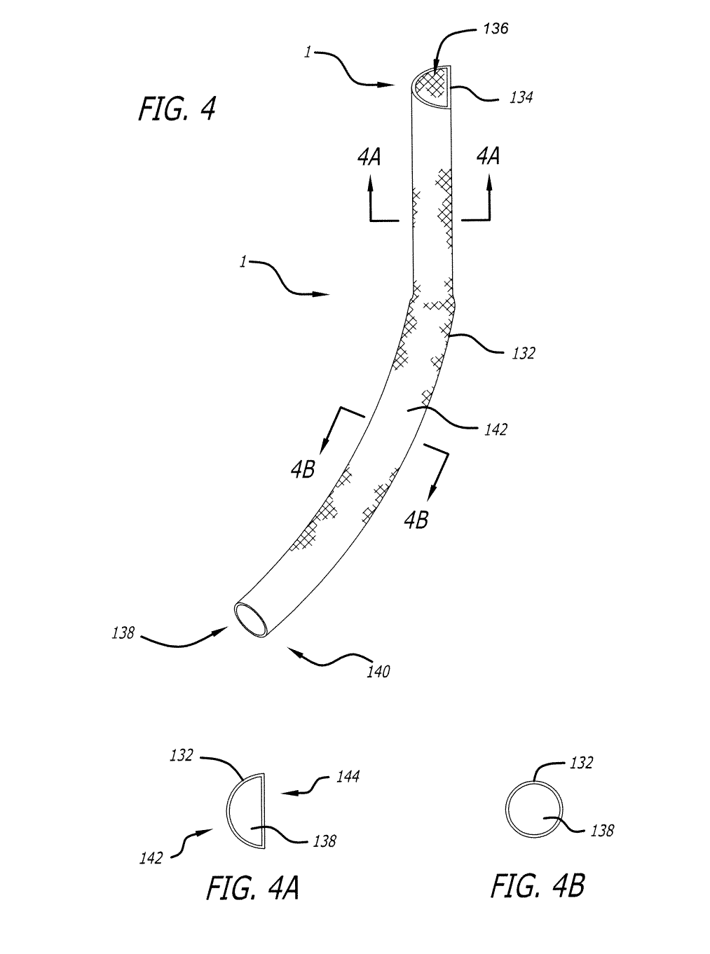

According to embodiments, for example two independent stent grafts 1 (as shown in FIG. 4) with D-shaped proximal ends and round distal ends are used to form the endovascular graft when two flat sides of the grafts face against each other FIG. 2C. In operation, each D-shaped graft may be loaded in the sheath of a delivery apparatus so that the first D-shaped graft can be accurately deployed in a mated fashion against the second D-shaped graft. According to embodiments, the grafts are inserted into the aorta via bilateral femoral sheaths and simultaneously deployed FIGS. 2B & 2C. The grafts may be rotated to align the flat sides against each other and mate. The flat side of the D-shape may incorporate a radiopaque marker (1am) fabricated from a platinum wire or other radiopague like material. The marker is positioned at an angle relative to each D-shaped portion that when a pair are aligned and “X” becomes visible. In other words, when visualized under fluoroscopy the markers of the two grafts align in parallel when the D's are properly effaced, each marker 1am forming one half of said “X”.

D-grafts 1 allow a non-custom method of supra and infra renal EVAR by separating treatment of each renal artery area. Position of the grafts can be independently adjusted up or down to the height of the renal ostia to accommodate varying anatomy. Complete EVAR can be performed with only two components selected for diameter (proximal and distal), length and renal ostia when desired.

According to embodiments, for example, D-shaped stent grafts 1 of the present invention form a cylindrical-like tubular appearance when two flat sides of the grafts are emplaced as they face each other or mate intimately against each other as in FIG. 2C. In embodiments, the graft is formed of ultrathin low or zero porosity PTFE which encases a braided Nitinol® wire stent frame. The PTFE is layered and sintered to encase the frame and thermally processed so that it is capable of elongating when the braided frame is compressed and inserted into the delivery catheter. In further embodiments, the graft is formed from a corrugated/ribbed polyester fabric material (for example, Dacron) or other suitable material, which encourage select endotheliazation outside of the sealing described above and claimed below. According to embodiments, the D-graft comprises openings (through the cells of the braids) for blood flow into a renal artery, wherein the opening may be created prior to implantation or be created by a wire piercing after the D-graft is placed in-situ, followed optionally by balloon expansion, as known to those skilled in the art.

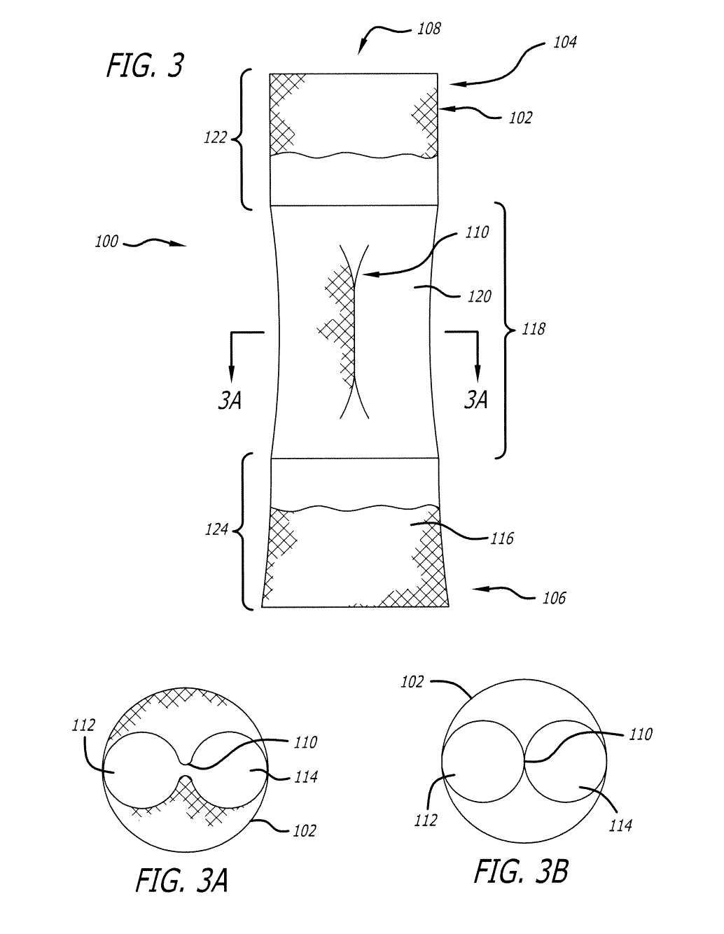

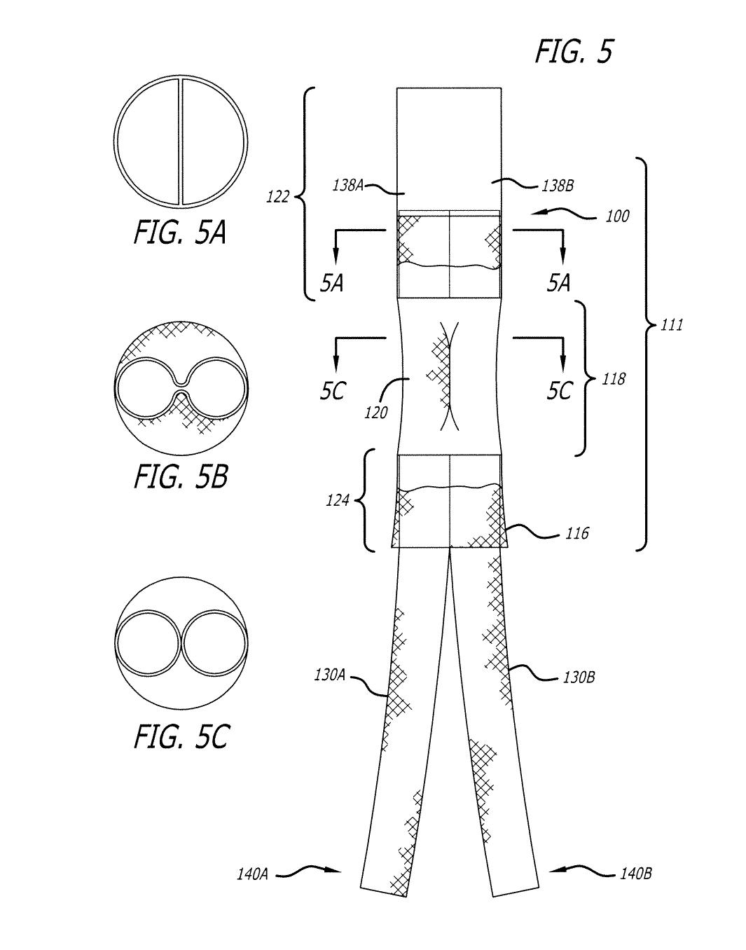

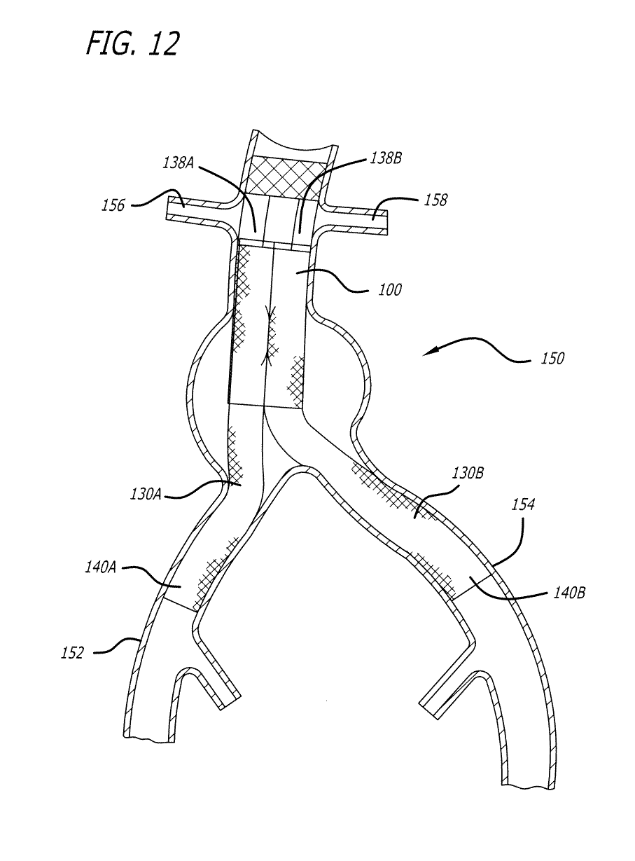

Referring to FIG. 3 and FIG. 5 D-shaped grafts 1 may be placed within another stent graft or aortic cuff 100 which is first placed and positioned within the infra and trans-renal segment of the aorta as shown in FIG. 3 & FIG. 12. The aortic cuff is placed first to provide a structure or frame to straighten out and reinforce an angulated or tortuous aortic neck and to provide for additional infra-renal aortic sealing. Referring to FIG. 3, there is illustrated a side elevational view of an aortic trunk or cuff 100, configured for endolumenal advancement into the aorta as will be discussed. The cuff 100 comprises a tubular body 102, extending between a superior end 104 and an inferior end 106. A central lumen 108 extends throughout the length of the cuff 100. Primary stent assembly 111 is that constructed to house the other subcomponents.

The central lumen 108 is optionally divided by a divider 110 into a first flow path 112 and a second flow path 114. As illustrated in cross sectional view in FIGS. 3A and 3B, the first flow path 112 and second flow path 114 may be either completely (FIG. 3B) or partially (FIG. 3A) isolated from each other.

The tubular body 102 may comprise a wire weave 116, utilizing any of a variety of metal or polymeric wires or filaments depending upon the desired clinical performance. In one implementation of the invention, the wire weave comprises a nickel titanium alloy having a diameter of no more than about 0.020 inches, and preferably no more than about 0.0010 inches. In one implementation of the invention, the wire has a diameter of approximately 0.009 inches and braided into a diamond shape with a diameter of approximately 0.160 inch with intersecting angles of 30 degrees. Alternatively the tubular body 102 can be laser cut from a metal tube such as Nitinol® then expanded and heat set into the desired final configuration.

In the vicinity of a central zone 118, the tubular body 102 is provided with a seal-supporting fabric layer 120 which overlaps on the outside of primary stent assembly 118, for redundant or supplemental sealing and endothelialization purposes ads described above and below and claimed hereafter. The central zone 118 is positioned between a superior zone 122 and an inferior zone 124. The overall length of the tubular body 102 may be varied considerably, depending upon the desired clinical performance and intended patient population. In general, tubular body 102 will have an axial length of at least about 40 mm and not more than about 80 mm. Typically, the axial length of tubular body 102 will be within the range of from about 45 mm to about 65 mm. The axial length of the central zone 118, and thus the axial length of the impermeable layer 120 will typically be at least about 10% and often at least about 20% of the overall length of the tubular body 102. In one embodiment, the tubular body 102 is approximately 60 mm in length, and the central zone 118 is approximately 15 mm in length.

Referring to FIG. 4, there is illustrated an implementation of a D-graft in accordance with the present invention. The graft 130 comprises an elongate flexible tubular body 132 extending between a superior opening 134 at superior end 136 and an inferior opening 138 at inferior end 140. Tubular body 132 may comprise a wire or filament braid or weave, such as a Nitinol® wire, as has previously been discussed. The tubular body 132 preferably comprises an impermeable layer 142 which extends along at least about 50% and preferably at least about 75% of the length of tubular body 132. According to embodiments of the invention, the tubular body 132 has an axial length of at least about 170 mm and the impermeable layer 142 has an axial length of at least about 130 mm. The impermeable layer preferably has a sufficient axial length to reach from the renal artery to the wall of the iliac artery just proximal to the internal iliac artery at the inferior end. A section of uncoated wire may be provided at each of the inferior end 140 and superior end 136, which may facilitate endothelialization, as is understood in the art, thus further discussion of the same has been omitted.

Referring to FIG. 4A, a cross sectional configuration of the tubular body 132 in the vicinity of the superior end 136 is in the form of a semi-circle or “D” as has been described is depicted. In its implanted orientation, a lateral wall 142 has an arcuate configuration, which may be in the form of a substantially constant radius curve. The radius of the curvature is selected to cooperate with the anticipated inside diameter of the aorta, as will be apparent in view of the disclosure herein. A medial wall 144 is in the nature of a secant, or diameter, and is substantially planar in the transverse dimension to facilitate cooperation with a second iliac graft. The second iliac graft is not separately illustrated in FIG. 4, but is preferably a mirror image of the graft illustrated in FIG. 4.

The cross-sectional configuration of the graft 130 may be constant throughout its axial length. Alternatively, the cross-sectional configuration may transition into a substantially circular cross-section, such as is illustrated in FIG. 4B. A circular or substantially circular configuration for the tubular body 132 in the vicinity of the inferior end 140 facilitates sealing between the tubular body 132 and the corresponding iliac artery, as will be appreciated by those of skill in the art.

The inferior zone 124 is generally at least about 15 mm and preferably within the range of from about 5 mm to about 10 mm in length. The length of the superior zone 122 is generally at least about 25 mm and preferably within the range of from about 15 mm to about 35 mm.

The permeable/endotheliazation layer 120 may comprise any of a variety of materials described previously herein, depending upon a variety of factors such as thrombogenicity, porosity and the desired crossing profile of the deployment catheter. In one implementation of the invention, impermeable layer 120 comprises ePTFE, having a wall thickness of no more than about 0.004 inch. Dacron and any of a variety of other ultrathin materials may alternatively be utilized.

The aortic cuff/primary stent assembly 110, 111, 118 are configured to cooperate with a first and second independently deployable D-grafts or sleeves, to produce a formed in situ bifurcation graft. Alternatively the D-grafts can also be circular grafts deployed within the circular segment 120 of cuff 100.

Referring now still also to FIG. 5, there is illustrated a schematic view of an assembled modular abdominal aortic aneurysm graft in accordance with this aspect of the present invention. As assembled, the first iliac D-graft 130 extends axially through the first flow path 112, such that the superior end 136 of iliac graft 130 is aligned approximately with the superior of the aortic cuff mother-stent assembly 100, 111. The iliac graft 130A is rotationally aligned with respect to the aortic cuff 100 such that the medial wall 144 faces, and preferably is in contact with the divider 110.

A second iliac D-graft 130B extends axially through the second flow path 114. Second iliac graft 130B may be aligned in a mirror image fashion with respect to first iliac graft 130A. Alternatively, iliac graft 130B may be positioned higher or lower in the superior inferior axis than the first iliac graft 130A. Thus, the superior end of a first iliac graft may be positioned at least about 0.5 cm, in some assemblies at least about 1 cm, and in certain applications at least about 2 cm higher than the superior end of a second iliac graft. This customization may be utilized to accommodate dissimilar locations (levels) of the renal arteries when considered along the superior inferior axis, and increase the sealing area as described infra.

In embodiments of the invention, material 120 of aortic cuff 100 is constructed from polyester and the D-graft 130 covering is constructed from ePTFE, Dacron, or combinations of the materials.

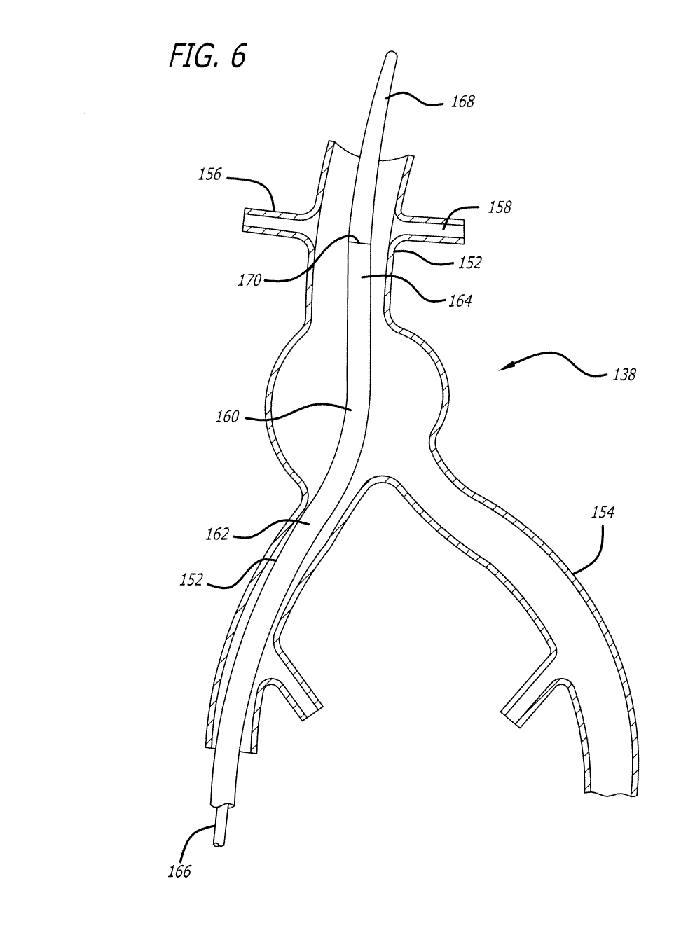

Assembly of the modular abdominal aortic aneurysm graft in accordance with the present invention will be illustrated with reference to FIG. 6 through 12. Referring to FIG. 6, there is schematically illustrated the portion of the vascular anatomy containing an aneurysm 150 at the bifurcation of the aorta 151 into the ipsilateral iliac 152 and contralateral iliac 154. A first renal artery 156 and second renal artery 158 are also illustrated, although other arteries have been omitted for simplicity. The anatomy illustrated in FIG. 6 is highly schematic, and subject to considerable variation from patient to patient with respect to both the angular relationship and launch points of the renal and iliac arteries with respect to the longitudinal axis of the aorta as well as with respect to the shape and location of the aneurysm 138.

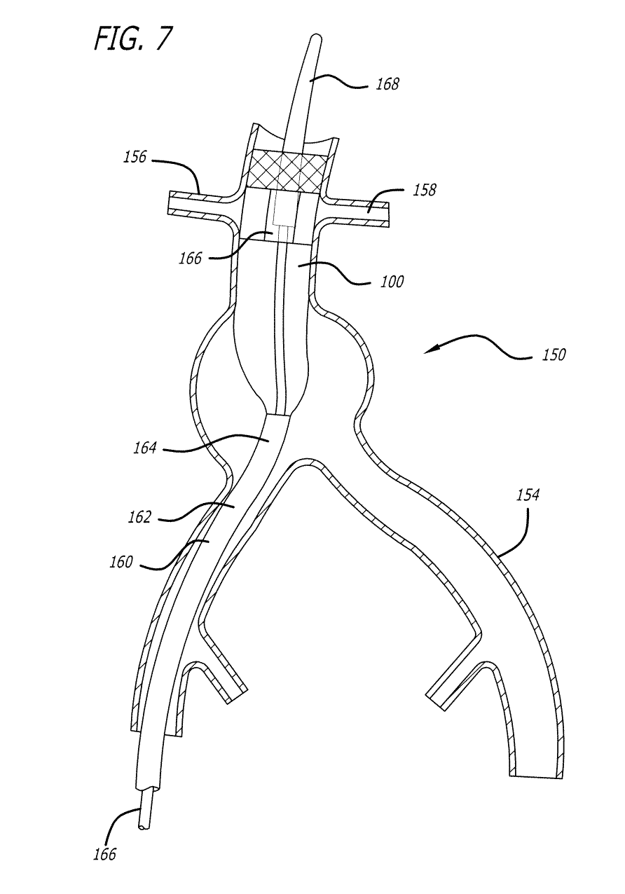

A deployment catheter 160 is illustrated spanning the aneurysm 138. Deployment catheter 160 is positioned using conventional techniques which will not be described in detail herein. In general, a guidewire having an outside diameter typically within the range of from about 0.025 to about 0.035 is percutaneously inserted into the arterial system such as at the femoral artery. The guidewire is advanced superiorly through the corresponding iliac toward the aorta, and advanced to the level of the renal arteries or higher. The deployment catheter 160 is thereafter advanced over the wire into the position illustrated in FIG. 6 and FIG. 7.

Deployment catheter 160 comprises an elongate flexible tubular body 162 having a proximal end 164. An elongate flexible support tube 166 extends axially throughout the length of the tubular body 162 which carries a nose cone or other blunt tip 168. A part line 170 separates the nose cone 168 from the tubular body 162, and one or more radiopaque markers is carried by one or more of the nose cone 168, tubular body 162 and support tube 166 to facilitate navigation under fluoroscopic guidance to the desired deployment site. Typically, the deployment catheter 160 will be percutaneously introduced and translumenally advanced to approximately the position illustrated in FIG. 6, with the part line 170 in the vicinity of and typically slightly superior to the renal arteries.

As illustrated in FIG. 7, the deployment catheter 160 is manipulated such that the tubular body 162 is distally retracted relative to the support tube 166. This allows the nose cone 168 to retain its initial position, while the proximal end of the tubular body 162 is proximally retracted opening the catheter at the part line 170 as illustrated.

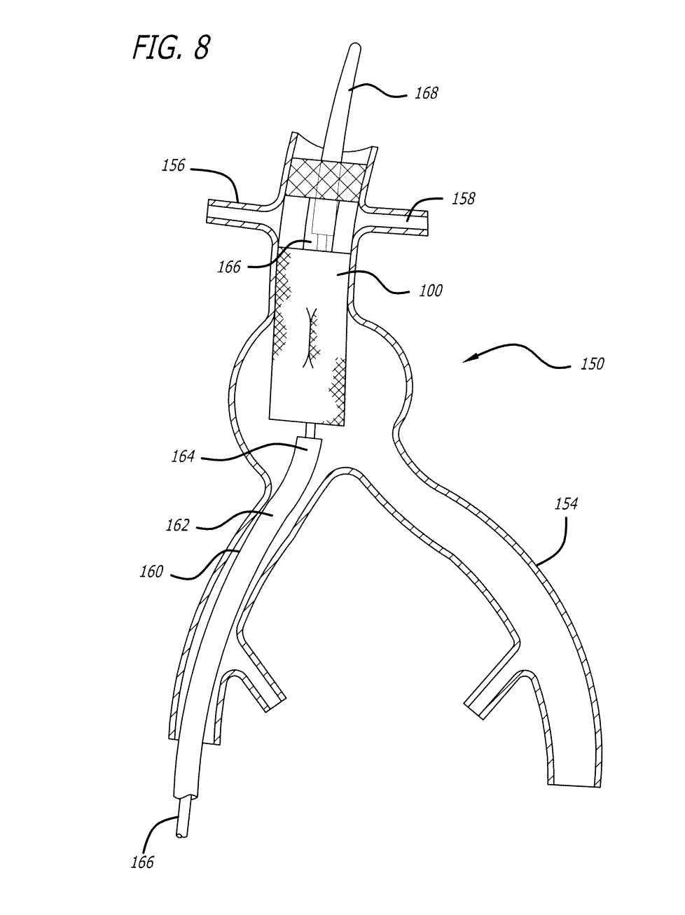

The aortic cuff 100 is radially compressed and constrained within the distal end 164 of the tubular body 162. Proximal axial retraction of the tubular body 162 relative to the support tube 166 gradually exposes the aortic cuff 100. Aortic cuff 100 radially outwardly expands under its inherent bias, until encountering resistance to further expansion provided by the wall of the aorta. Prior to full deployment of the aortic cuff 100 the cuff can be recaptured by catheter 164 and repositioned if necessary so that the distal end of impermeable segment 118 is positioned just below the lowest renal artery and above the aneurysm within the healthy neck of the aorta. Proximal retraction of the tubular body 162 is continued until, as illustrated in FIG. 8, the aortic cuff 100 is fully deployed from the deployment catheter 160 and anchored within the aorta. The tubular body 162 may thereafter be axially distally advanced along the support the tube 166 back into contact with the proximal end of the nose cone 168, to provide a smooth exterior surface. Deployment catheter 160 may thereafter be proximally retracted from the patient with the guide wire left in place.

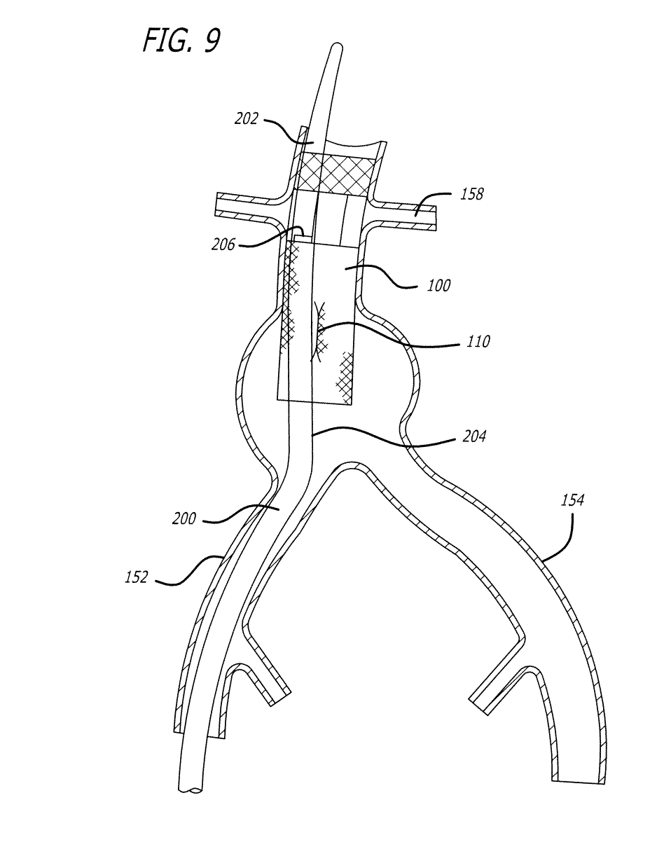

Referring to FIG. 9, an ipsilateral D-graft deployment catheter 200 may thereafter be introduced such as via the femoral artery, and advanced translumenally through the ipsilateral iliac and also through the first flow path 112 of the aortic cuff 100. The ipsilateral iliac D-graft deployment catheter 200 is similar to the deployment catheter 160 previously discussed, and includes a distal nose cone 202 axially aligned with a tubular body 204, and separated therefrom by a part line 206. The ipsilateral iliac graft (not illustrated) has previously been radially reduced such as by compression and constrained within the tubular body 204.

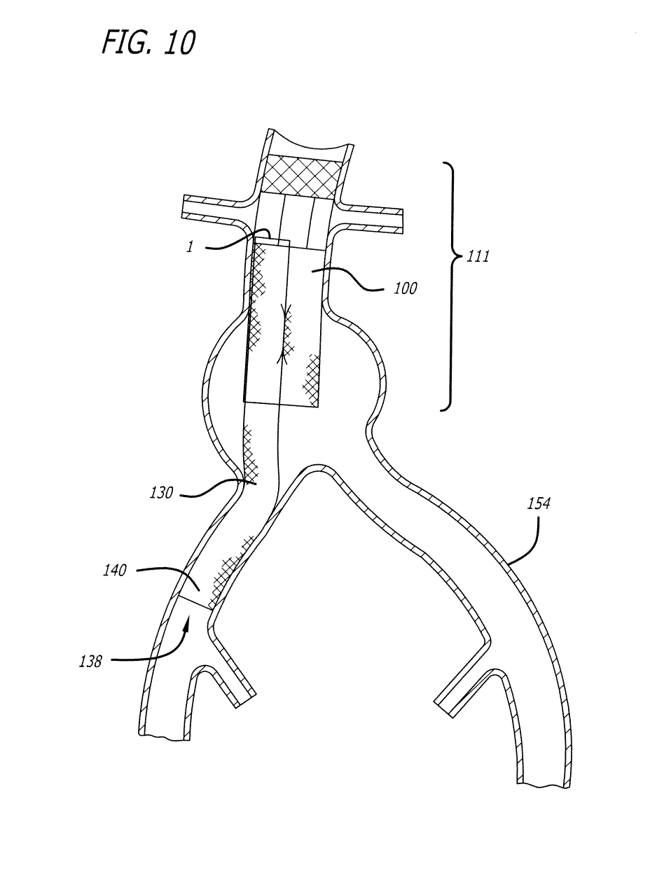

The tubular body 204 is thereafter proximally retracted relative to the distal nose cone 202, thereby separating the outside sidewall of the catheter at the part line 206 and exposing the ipsilateral iliac D-graft. Proximal retraction of the tubular body 204 along an axial length greater than the length of the iliac graft exposes the iliac graft and allows it to fully radially expand. If necessary, as the D segment is deployed, tubular body 204 can be advanced to recapture the stent graft for repositioning. As the D segment is deployed the catheter is rotated so that the D segment is aligned within segment 102 and within the limits set by the inside diameter of the first flow path 112 within aortic cuff 100 at the superior end and the diameter of the iliac artery at the inferior end. The ipsilateral iliac graft deployment catheter 200 may thereafter be proximally withdrawn from the patient, leaving the partially assembled construct as illustrated in FIG. 10.

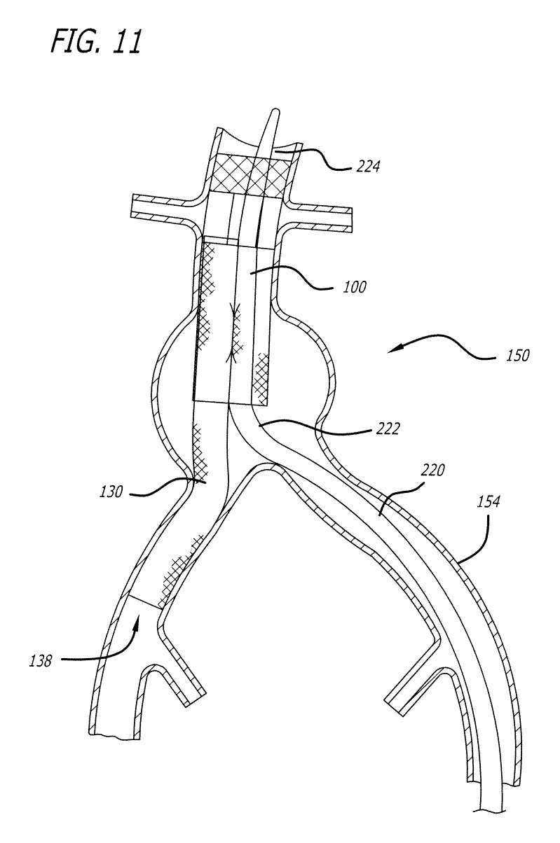

A contralateral femoral access is then provided, and a guidewire advanced via the contralateral femoral and iliac pathways and through the second flow path 114 in aortic cuff 100. A contralateral iliac graft deployment catheter 220 is thereafter translumenally advanced over the wire and into the position schematically illustrated in FIG. 11. Proximal retraction of an outer tubular sleeve 222 relative to a distal nose cone 224 exposes the contralateral iliac D-graft 130B, which radially outwardly expands to provide a seal with the first deployed D-graft and the second flow path 114 of aortic cuff 100 at the superior end, and with the contralateral iliac wall at the inferior end. The contralateral graft deployment catheter 220 is thereafter distally withdrawn, leaving the assembled abdominal aortic aneurysm graft construct as illustrated in FIG. 12.

Grafts constructed in accordance with the present invention are believed to enable the construction of an endovascular straight segment or bifurcation graft utilizing a catheter which can have a lower crossing profile than those conventionally found in the prior art. For example, the braided construction of the wire support allows a degree of axial elongation and radial compression which permits the compressed graft to be loaded within a smaller deployment catheter than a wire frame constructed in a conventional “Z stent” configuration. In general, bifurcation grafts in accordance with the present invention are preferably dimensioned such that they can be placed in an aorta having a diameter of at least about 25 mm, via an access catheter having a diameter of no more than about 12 Fr. In one implementation of the invention, the bifurcation graft may be implanted in an aorta having a diameter of at least about 25 mm, using a deployment catheter having a diameter of no more than about 12 French. This implementation of the invention has an aortic cuff which expands to an average outside diameter of at least about 25 mm in an unconstrained expansion.

Generally, the aortic cuff 100 delivered from a 12 French catheter will have an unconstrained expansion to a diameter of at least about 20 mm, and preferably at least about 27 mm. Primary stent assembly 111 likewise may be customized per the anatomy of the patient.

The present invention additionally permits customization of the graft to optimize the overlap of the superior end of the graft with healthy tissue in the aorta, without jailing the renal arteries. This may be desirable in patients having a first renal artery which opens into the aorta at a first level evaluated along the direction of blood flow, and a second renal artery opening into the aorta at a second, different level which may be lower or farther downstream than the first level. A first iliac D-graft may be deployed such that the superior end resides inferiorly to the second level, and the graft is on the second level side of the cuff. The second iliac graft may be implanted with a superior end at a higher level such that it is just inferior to the first renal artery, and offset from the superior end of the first iliac graft by at least about 0.5 cm, at least about 1.0 cm, in some instances at least about 2.0 cm.

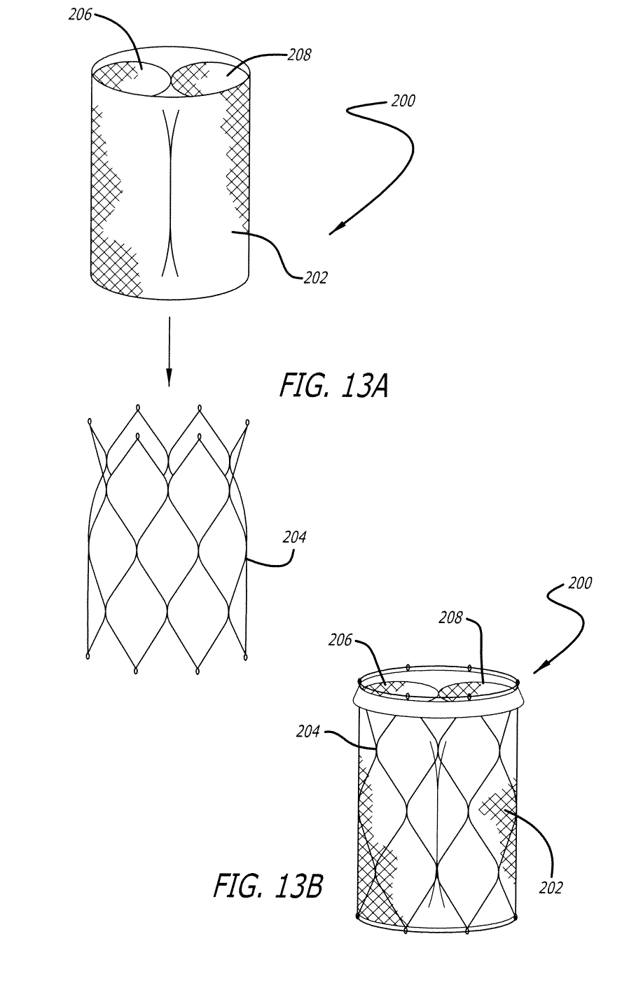

Referring now to FIGS. 13A and 13B, details of the shotgun neckframe 200 show now lumen splitting allows users to accommodate different anatomies. When used as a subrenal device shotgun neckframe 200 can be repositioned after deployment with fabric 202 and stent material 204 providing redundant sealing for apertures 206 and 208 which accommodate D-shaped endografts.

The present invention has been described and illustrated in connection with certain specific embodiments thereof. However, it will be understood by those skilled in the art that various changes in form and details may be made therein without departing from the scope and spirit of the invention. For all of the embodiments described above, the various elements and variables may be interchanged, and the steps of the method may be interchanged, without departing from the present invention.

What is claimed is:

1. A bifurcated endograft for aneurysm treatment comprising, in combination: a system which can be delivered percutaneously through a 12 Fr. or less vascular introducer further comprising; at least an endograft element capable of being, disposed within a primary stent assembly inside of the aorta.

2. The bifurcated endograft of claim 1, further comprising at least two endograft units capable of being disposed within a primary stent assembly.

3. The bifurcated aortic endograft of claim 1, comprising a plurality of separate endografts, each endograft having a lumen, a proximal end and a distal end, each endograft further comprises a partially covered flexible tubular braided wire frame having a proximal end with a generally D-shaped cross-section configured to be secured against a second D-shaped graft to form a circular graft within the infrarenal aorta; and, each having a distal end with a generally circular cross section configured to be placed and fixed in each of the iliac arteries.

4. The bifurcated endograft of claim 2, further comprising three components, an infrarenal aortic stent intended to engage the aorta above and below the renal arteries; and containing a covered segment below the renal arteries which serves to seal the infrarenal neck and engage and constrain two endografts with a generally D-shaped configuration at the proximal end; and a circular configuration at the distal end for placement in the iliac arteries.

5. A modular two-piece abdominal aortic endograft system with D-shaped proximal ends and circular distal ends which can be axially aligned within the aorta; wherein each said modular piece is independently adjustable up and down relative to each other to accommodate the naturally anatomically variable orientation of the renal arteries.

6. A method of constructing a modular quasi-customizable endovascular graft in situ, comprising in combination the steps of:
translumenally advancing a first deployment catheter to access the aorta;
deploying a tubular cuff within the aorta;
translumenally advancing a second deployment catheter to access the tubular cuff from the bottom deploying a first iliac graft within in the tubular cuff;
translumenally advancing a third deployment catheter to access the tubular cuff the bottom; and,
deploying a second iliac graft within the tubular cuff

7. The method of claim 6, wherein the tubular cuff expands from a constrained to an unconstrained expansion diameter.

8. The method of claim 7, wherein the first and second deployment catheters are introduced through the same access point.

9. The method of claim 8, wherein the cuff has a substantially circular cross section at a first location along its axial length, and first and second side by side flow channels at a second location along its length.

10. The method of claim 9, wherein the deploying a first iliac graft within the tubular cuff comprises deploying the first graft within the first flow channel such that a substantially flat side of the first graft faces the second flow channel.

11. The method of claim 10, where in the deploying a second iliac graft within the tubular cuff comprises deploying the second graft within the second flow channel such that a substantially flat side of the second graft faxes the substantially flat side of the first graft.

12. A low-profile modular endograft system comprising, in combination: a cuff and at least two endograft units, each endograft unit having a lumen, a proximal end and a distal end, wherein each endograft unit comprises a flexible tubular woven wire frame having a proximal end with a generally D-shaped cross-section configured to be secured at the cuff and a distal end having a generally circular cross section-configured to be placed and fixed in each of the iliac arteries, and a seal between each of said endograft units and said cuff

13. The low-profile modular endograft system of claim 12, the cuff and related structures being an aortic cuff

14. The low-profile modular endograft system of claim 13, capable of being introduced through an introducer profile of at least 12 Fr.

15. The low-profile modular endograft system of claim 14, each said endograft unit is a braided stent like device with having an optimized braid angle of at least about 45 degrees or greater.

16. The low-profile modular endograft system of claim 15, further comprising fabric layers to accommodate for lengths of foreshortening.

17. The low-profile modular endograft system of claim 16, further comprising a fabric shotgun-shaped prosthetic having flared proximal and distal ends.

18. The low-profile modular endograft system of claim 17, further comprising barbs-which are sized and configured to allow the graft to move in an advancing direction, whereby said barbs engage the vessel wall in which emplaced when the graft units moves in a reverse direction.

19. The low-profile modular endograft system of claim 18, further comprising septal bowing said D-shaped endografts to promote sealing.

20. The low-profile modular endograft system of claim 19, further comprising septal angled radiographic markers to facilitate imaging and placement of each said endograft unit.
